(19) United States
(12) Patent Application Publication
(10) Pub. No.: US 2024/0298906 A1
(43) Pub. Date: Sep. 12, 2024

(54) PULSE WAVE DETECTOR

(71) Applicant: Mitsubishi Electric Corporation, Tokyo (JP)

(72) Inventors: Ryohei MURACHI, Tokyo (JP); Yudai NAKAMURA, Tokyo (JP)

(73) Assignee: Mitsubishi Electric Corporation, Tokyo (JP)

(21) Appl. No.: 18/281,958

(22) PCT Filed: Mar. 26, 2021
(86) PCT No.: PCT/JP2021/012915

§ 371 Date: Sep. 14, 2023

(51) Int. Cl. A61B 5/024

(52) U.S. Cl. CPC A61B 5/02416

(57) ABSTRACT

A pulse wave detection device according to the present disclosure technology is a pulse wave detection device that detects a pulse wave from an image obtained by imaging a subject, and includes an inter-region data adjusting unit including a phase calculating unit and a phase correcting unit. The phase calculating unit calculates a phase difference between a plurality of time-series luminance signals of a region of interest of the image, and the phase correcting unit corrects a phase of the time-series luminance signal on the basis of the calculated phase difference.

### TECHNICAL FIELD

The present disclosure technology relates to a pulse wave detection device.

### BACKGROUND ART

A technology for measuring a pulse rate of an examinee from an image obtained by imaging the face of the examinee is known. For example, it is known that a pulse wave signal corresponding to a heartbeat can be extracted from a video signal obtained by imaging an ROI (hereinafter referred to as a “region of interest”) of a face using independent component analysis.

Furthermore, for example, Patent Literature 1 discloses a technology capable of performing accurate measurement even when the face of an examinee moves or even when light incident on the face changes.

A measurement device according to Patent Literature 1 solves a linear BSS problem of estimating “signals primarily contributed by skin melanin” and “signal contributed by hemoglobin in blood vessel (hereinafter referred to as a “PG signal”)” from a pair of luminance signals of each sample using a pair of small regions selected from a region of interest as a sample, and extracts a PG signal.

### CITATION LIST

### Patent Literature

Patent Literature 1: JP 2017-93760 A

### SUMMARY OF INVENTION

### Technical Problem

In the related art exemplified in Patent Literature 1, it is assumed that the facial skin of the examinee is sufficiently exposed even if the subject wears glasses or the like. Thus, the small region selected from the region of interest is typically the left and right cheeks.

Recently, there is a demand for detecting a pulse wave signal of an examinee even from an image obtained by imaging the face of the examinee wearing a mask. When the image analysis technology is used, even in the conventional pulse wave detection device, it may be possible to select a small region where the skin is exposed from the region of interest. In this case, the small region chosen as a sample may have a different distance from the heart, such as a pair of forehead region and neck region. The fact that the distances from the heart are different means that the distance at which the pulse wave propagates is different, which means a shift on the time axis of the pulse wave signal in each small region. When a pair of signals having a shift on the time axis is used in this manner, extraction of the pulse wave component ends in failure in the independent component analysis or main component analysis.

An object of the present disclosure technology is to solve the above problem and to provide a pulse wave detection device that detects a pulse wave signal of an examinee even from an image obtained by imaging the face of the examinee wearing a mask.

### Solution to Problem

A pulse wave detection device according to the present disclosure technology is a pulse wave detection device for detecting a pulse wave from an image obtained by imaging a subject, and includes an inter-region data adjusting unit including a phase calculating unit and a phase correcting unit. The phase calculating unit calculates a phase difference between a plurality of time-series luminance signals of a region of interest of the image, and the phase correcting unit corrects a phase of the time-series luminance signal on the basis of the calculated phase difference.

### Advantageous Effects of Invention

The pulse wave detection device according to the present disclosure technology has the above configuration, and a phase between a plurality of time-series luminance signals of a region of interest is corrected even when an examinee wears a mask. By the operation of correcting the phase, the pulse wave detection device according to the present disclosure technology can detect a pulse wave signal of an examinee even from an image obtained by imaging the face of the subject wearing a mask.

### BRIEF DESCRIPTION OF DRAWINGS

FIG. 1 is a schematic diagram illustrating a problem to be solved by the present disclosure technology.

FIG. 2 is a schematic diagram illustrating a principle of calculating a pulse rate from an image obtained by imaging a face of an examinee not wearing a mask by a pulse wave detection device according to a related art.

FIG. 3 is a block diagram illustrating functional blocks of a pulse wave detection device (100) according to a first embodiment.

FIG. 4 is a schematic diagram illustrating processing steps of skin region performed by a skin region detection unit (10) of the pulse wave detection device (100) according to the first embodiment.

FIG. 5 is a schematic diagram illustrating processing steps performed by a cover detection unit (20) of the pulse wave detection device (100) according to the first embodiment.

FIGS. 6A and 6B are schematic diagrams illustrating processing steps performed by a measurement region setting unit (30) of the pulse wave detection device (100) according to the first embodiment. FIG. 6A is a schematic diagram illustrating a skin region S(k) before the measurement region setting unit (30) performs processing. FIG. 6B is a schematic diagram illustrating the skin region S(k) after the measurement region setting unit (30) performs the processing.

FIG. 7 is a schematic diagram illustrating that a phase of a pulse wave is shifted between a forehead and a neck.

FIG. 8 is a schematic diagram illustrating an operation of a pulse wave estimating unit (60) of the pulse wave detection device (100) according to the first embodiment.

FIG. 9 is a schematic diagram illustrating an operation of a pulse wave estimating unit (60) of a pulse wave detection device (100) according to a second embodiment.

FIG. 10 is a flowchart illustrating a flow of the pulse wave detection device (100) according to the present disclosure technology.

FIG. 11 is a block diagram illustrating a configuration of an inter-region data adjusting unit (50) of a pulse wave detection device (100) according to a third embodiment.

### DESCRIPTION OF EMBODIMENTS

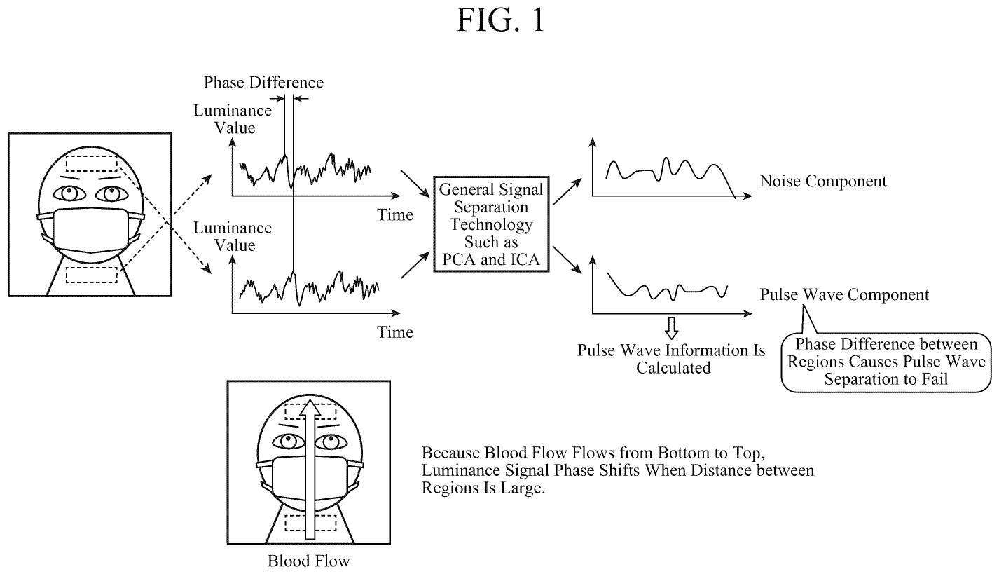

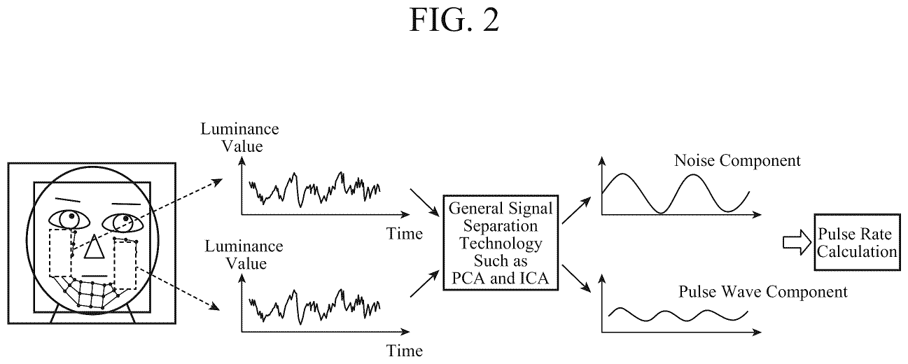

A difference between a pulse wave detection device (100) according to the present disclosure technology and the prior art will be apparent from the following description supplemented with the related art. FIG. 1 is a schematic diagram illustrating a problem to be solved by the present disclosure technology. FIG. 2 is a schematic diagram illustrating a principle of calculating a pulse rate from an image obtained by imaging the face of an examinee not wearing a mask by a pulse wave detection device according to the related art.

The leftmost image in FIG. 2 represents an image of the face of an examinee not wearing a mask. The pulse wave detection device according to the related art sets a region of interest (a region surrounded by a white square in the drawing) from the image of the face of the examinee, and selects regions of left and right cheeks as a pair of small regions as a sample.

The second block from the left in FIG. 2 is a graph representing luminance values of left and right cheek regions selected as samples on a time axis.

The third block from the left in FIG. 2 represents a block in which independent component analysis or main component analysis is performed. In this block, signal processing is performed on a signal of a pair of luminance values selected as a sample, and a pulse wave component and a noise component are extracted.

A fourth block from the left in FIG. 2 is a graph illustrating each of the extracted pulse wave component and noise component on the time axis.

The rightmost block in FIG. 2 represents a block for calculating the pulse rate from the signal of the extracted pulse wave component.

### First Embodiment

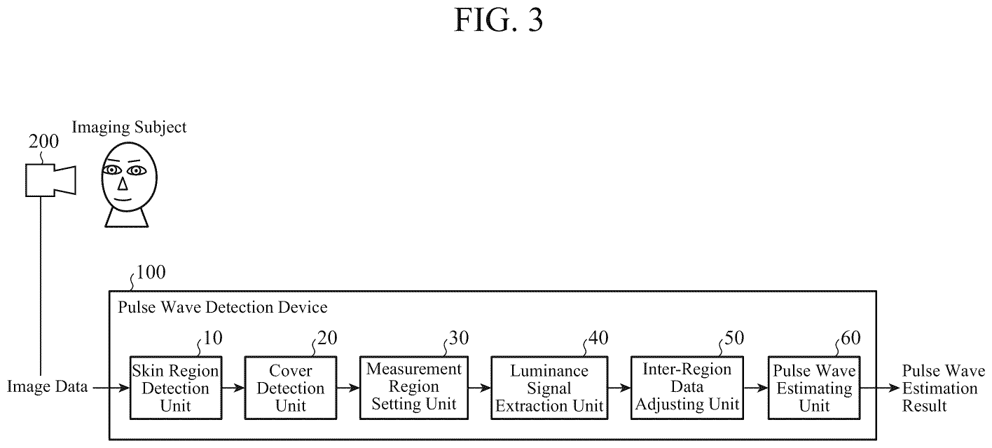

FIG. 3 is a block diagram illustrating functional blocks of a pulse wave detection device 100 according to a first embodiment. As illustrated in FIG. 3, the pulse wave detection device 100 includes a skin region detection unit 10, a cover detection unit 20, a measurement region setting unit 30, a luminance signal extracting unit 40, an inter-region data adjusting unit 50, and a pulse wave estimating unit 60. Furthermore, the pulse wave detection device 100 is connected to a camera 200.

The pulse wave detection device 100 may be an on-vehicle device, and the camera 200 may be a driver monitoring system (hereinafter referred to as “DMS”). In the related art, a subject as a target of detecting a pulse wave is referred to as a “examinee”, but in the present disclosure technology, a wider expression “subject” is used.

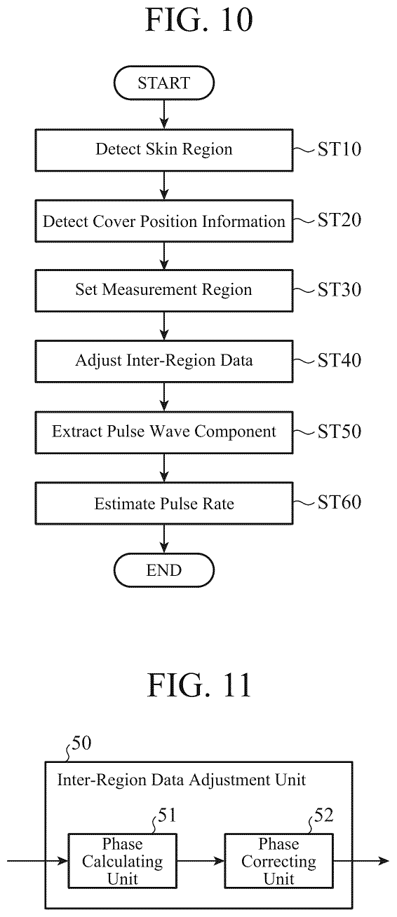

FIG. 10 is a flowchart illustrating a flow of the pulse wave detection device 100 according to the first embodiment.

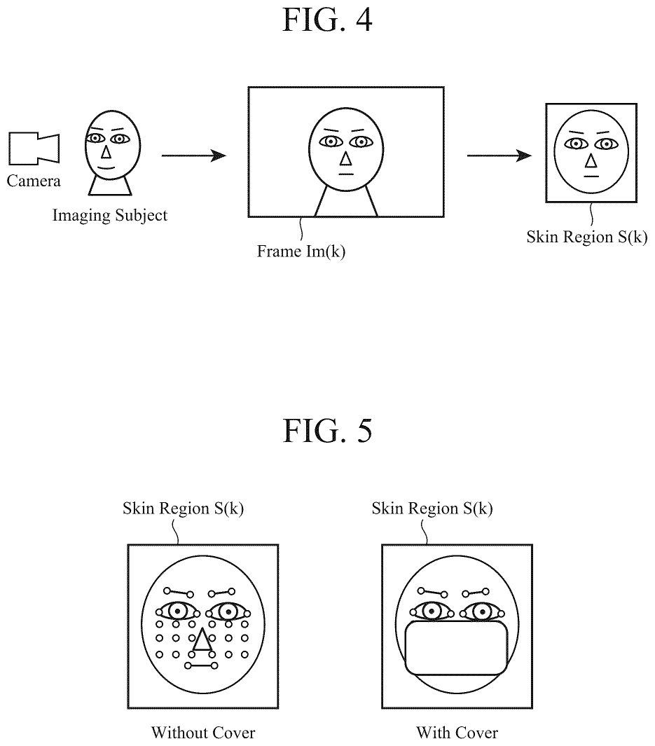

FIG. 4 is a schematic diagram illustrating processing steps performed by the skin region detection unit 10 of the pulse wave detection device 100 according to the first embodiment. Image data of the subject imaged by the camera 200 is input to the skin region detection unit 10. The skin region detection unit 10 detects a skin region S(k) which is a region including human skin from a frame Im(k) of the input image data (step indicated by ST10 in FIG. 10). Here, k represents a frame number.

As illustrated in FIG. 4, the skin region S(k) is a partial region of the frame Im(k), and may be, for example, a rectangle. More specifically, the skin region S(k) is a region obtained by trimming the frame Im(k), leaving a region of a portion of exposed skin represented by a face. Furthermore, the number of skin regions S(k) is not necessarily one, and may be a plurality of regions. The skin region S(k) detected by the skin region detection unit 10 is output to the cover detection unit 20.

FIG. 5 is a schematic diagram illustrating processing steps performed by the cover detection unit 20 of the pulse wave detection device 100 according to the first embodiment. The cover detection unit 20 determines whether or not the skin of the subject is covered by a mask or the like from the output skin region S(k). In a case where it is determined that the skin of the subject is covered, the cover detection unit 20 detects cover position information (step indicated by ST20 in FIG. 10).

For the detection of the cover position information, for example, pattern detection using a Haar-like feature amount or pattern detection using a HOG feature amount may be used. The cover detection unit 20 may refer to face organ point information of a face model without a cover and determine that there is a cover around an insufficient face organ point. The face organ point information indicates, for example, a point having features such as the inner corner of an eye and the outer corner of an eye in a case of an eye.

The detected cover position information is output to the measurement region setting unit 30 together with the frame Im (k) and the skin region S(k). Here, k is a serial number referred to as a frame number.

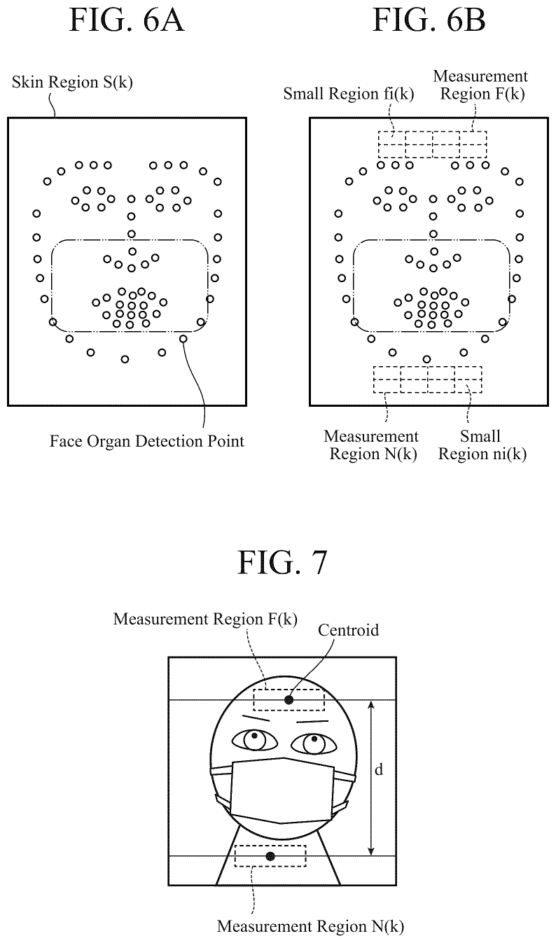

FIG. 6 is a schematic diagram illustrating processing steps performed by the measurement region setting unit 30 of the pulse wave detection device 100 according to the first embodiment. FIG. 6A is a schematic diagram illustrating a skin region S(k) before the measurement region setting unit 30 performs processing. FIG. 6B is a schematic diagram illustrating the skin region S(k) after the processing is performed by the measurement region setting unit 30.

The measurement region setting unit 30 sets a measurement region R (k) including a plurality of small regions rᵢ(k) for extracting a pulse wave signal from the skin region S(k) (step indicated by ST30 in FIG. 10).

The example illustrated in FIG. 6 illustrates a case where the cheek region is covered by the mask. The measurement region setting unit 30 sets, in the skin region S(k), a measurement region including eight small regions in each of a forehead portion and a neck portion. Here, the measurement region of the forehead portion is denoted as F(k), and the measurement region of the neck portion is denoted as N(k) so as to be distinguishable from each other. In addition, the eight small regions constituting the measurement region F(k) of the forehead portion are described as fᵢ(k). Similarly, the eight small regions constituting the measurement region N(k) of the neck portion are described as nᵢ(k). Here, the subscript i is a serial number attached to a small region.

In FIG. 6A, facial organ detection points are illustrated. For the detection of the coordinate values of the facial organ detection points, for example, a mathematical model such as a constrained local model (hereinafter referred to as “CLM”) may be used. Furthermore, a tracking technology such as a Kanade-Lucas-Tomasi (hereinafter referred to as “KLT”) tracker may be used for the measurement region setting unit 30. In addition, both CLM and KLT may be used. For example, the measurement region setting unit 30 may detect the coordinates of a facial organ point using CLM with respect to the skin region S(1) of the first frame Im (1) and track the facial organ point by KLT with respect to the skin region S (2) and the subsequent frames Im(2). In addition, for the purpose of eliminating the cumulative error of tracking, the CLM may be executed at a frequency of once in several frames for the KLT after two frames.

FIG. 6B illustrates an example in which the forehead portion and the neck portion are set as the measurement region, but the measurement region is not limited thereto. The portion set as the measurement region may be another portion such as the periphery of an eye, or may be any portion where the skin is exposed.

In addition, FIG. 6B illustrates an example in which the measurement region is divided into, but is not limited to, eight small regions.

Information regarding the measurement region set by the measurement region setting unit 30 and the small regions constituting the measurement region is output to the luminance signal extracting unit 40.

For each small region set by the measurement region setting unit 30, the luminance signal extracting unit 40 calculates a value representing luminance values of pixels included in the small region as luminance signal information Gᵢ(k). The representative value may be, for example, an average value of luminance values of pixels included in the small region. The representative value is not limited to the average value, and dispersion or the like may be used. The luminance signal extracting unit 40 connects the luminance signal information Gᵢ(k) for each k in time series to generate a time-series luminance signal.

The luminance signal extracting unit 40 may have a function of not only calculating the current k-th luminance signal information Gᵢ(k) but also storing the previous (k−1)-th luminance signal information Gᵢ(k−1) and calculating a difference between the k-th and (k−1)-th luminance signal information.

The time-series luminance signal in time series calculated by the luminance signal extracting unit 40 is output to the inter-region data adjusting unit 50 together with information of the measurement region from which the time-series luminance signal is obtained. Specifically, the information of the measurement region is information of which part of the face of the subject the measurement region is. In the example of FIG. 6, the information of the measurement region includes information that F(k) is the forehead and information that N(k) is the neck.

The inter-region data adjusting unit 50 processes a plurality of time-series luminance signals for each small region of each measurement region. An operation of the inter-region data adjusting unit 50 according to the first embodiment will be clear from the following description.

In general, in extraction of pulse wave information from an image, it is desirable to set a region of interest as wide as possible and use a luminance average value of the region of interest. Setting the region of interest wide leads to an improvement in the S/N ratio.

When this is applied to the present disclosure technology, it is desirable to perform the pulse wave information extraction by setting all the measurement regions set by the measurement region setting unit 30 as the region of interest. That is, it is desirable that the inter-region data adjusting unit 50 averages all the transmitted time-series luminance signals and extracts pulse wave information.

However, “to set a region of interest as wide as possible” also has a premise that a certain condition is satisfied. The certain condition is that the phases of the pulse waves are aligned in the region of interest. The extraction of the pulse wave information from the image using the independent component analysis or the main component analysis is also based on the premise that the phases of the pulse waves are aligned in the region of interest.

The problem to be solved by the present disclosure technology occurs when the phases of the pulse waves are not aligned in the region of interest. When the phases of the pulse waves are not aligned in the region of interest, the premise of the independent component analysis or the main component analysis is not satisfied, and the pulse wave information may not be accurately extracted.

Blood flows through the body by pumping action of the heart. Pulse waves are created by the heart. That is, if the distance of a path through which blood passes from the heart is equal, it can be said that the phases of the pulse waves are aligned. Conversely, if the distance of the path through which the blood passes from the heart is different, it can be said that the phases of the pulse waves are shifted.

In recent years, a situation in which a subject is wearing a mask is increasing. As described above, the mask limits the region of interest, and for example, the forehead and the neck are selected. Since the distance of the path through which blood passes from the heart is different between the forehead and the neck, the phases of the pulse waves are shifted. FIG. 7 is a schematic diagram illustrating that phases of pulse waves are shifted between the forehead and the neck. The phase difference here may be expressed as a delay time appearing on the time axis.

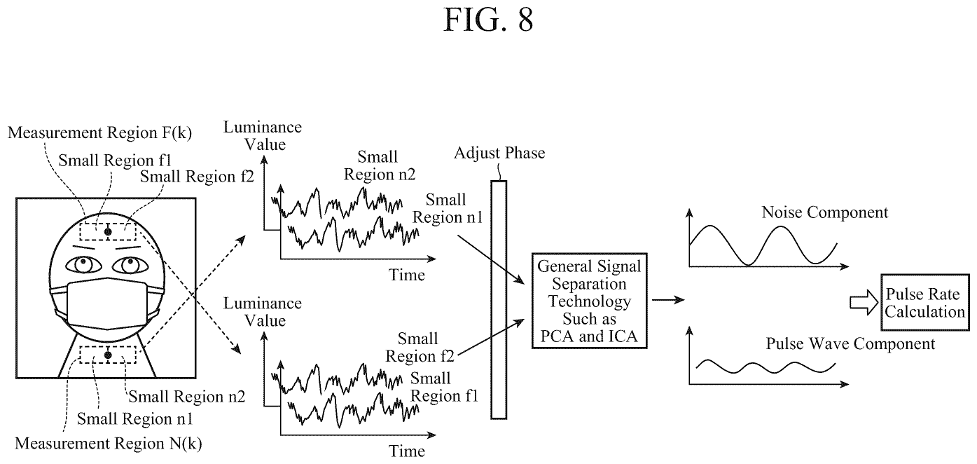

FIG. 8 is a schematic diagram illustrating the operation of the pulse wave estimating unit 60 of the pulse wave detection device 100 according to the first embodiment. As illustrated in FIG. 8, the inter-region data adjusting unit 50 according to the first embodiment adjusts phases between the plurality of time-series luminance signals on the basis of the information of the measurement region (step indicated by ST40 in FIG. 10).

The amount of phase to be adjusted, that is, the time shift amount of the time-series luminance signal can be obtained by several methods. The simplest method is a method of determining the time shift amount in such a way that the positions of the peaks of the time-series luminance signals are aligned.

The amount of phase to be adjusted may be obtained statistically. More specifically, the inter-region data adjusting unit 50 according to the first embodiment may hold information of the phase difference of pulse waves between face parts in advance. The information of the phase difference of pulse waves between face parts may be held in accordance with the feature of the subject. For example, the phase difference information may be held separately for gender and age. The face parts may be, for example, a forehead, an area under an eye, a cheek, a jaw, and a neck. The inter-region data adjusting unit 50 according to the first embodiment may adjust the phase between the plurality of time-series luminance signals on the basis of the information of the measurement region and the information of the phase difference held in advance.

The plurality of time-series luminance signals whose phases have been adjusted are output to the pulse wave estimating unit 60.

The pulse wave estimating unit 60 separates the plurality of time-series luminance signals whose phases have been adjusted into a pulse wave component and a noise component (step indicated by ST50 in FIG. 10). Independent component analysis or main component analysis may be used to separate the pulse wave component and the noise component. The pulse wave estimating unit 60 estimates the pulse rate from the extracted time-series data of the pulse wave component (step indicated by ST60 in FIG. 10).

As described above, since the pulse wave detection device 100 according to the first embodiment has the above configuration, the phase between the plurality of time-series luminance signals of the region of interest is adjusted even when the subject wears a mask. By operation of adjusting the phase, the pulse wave detection device 100 according to the first embodiment can detect the pulse wave signal of a subject even from an image obtained by imaging the face of the subject wearing a mask.

### Second Embodiment

In the pulse wave detection device 100 according to the first embodiment, the pulse wave estimating unit 60 adjusts phases between a plurality of time-series luminance signals on the basis of the information of the measurement region. A second embodiment has the same configuration as the first embodiment, but a pulse wave estimating unit 60 operates differently from the first embodiment to solve the problem of the present disclosure technology.

In the second embodiment, the same reference numerals as those used in the first embodiment are used unless otherwise specified. In the second embodiment, the description overlapping with the first embodiment is appropriately omitted.

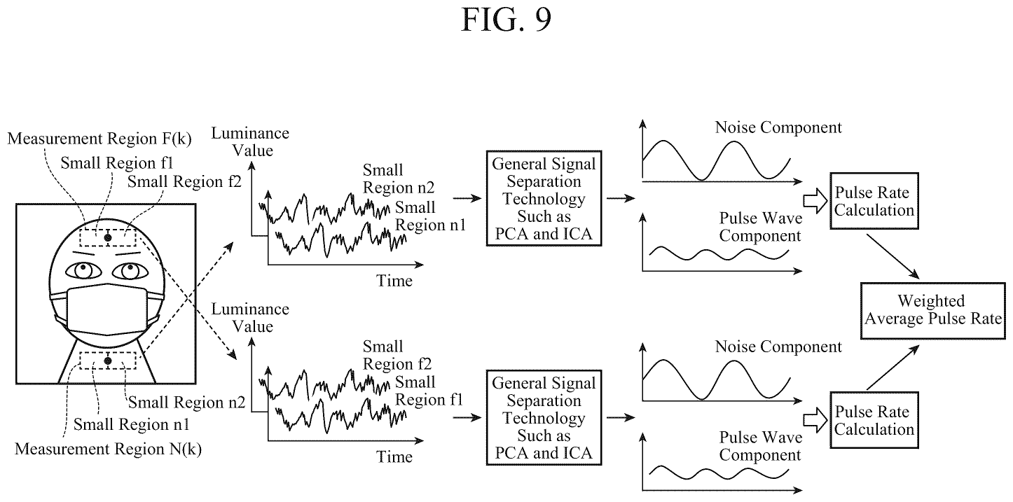

FIG. 9 is a schematic diagram illustrating the operation of the pulse wave estimating unit 60 of the pulse wave detection device 100 according to the second embodiment. As illustrated in FIG. 9, the inter-region data adjusting unit 50 according to the second embodiment groups the time-series luminance signals of the small region for each measurement region. Specifically, the inter-region data adjusting unit 50 according to the second embodiment instructs the pulse wave estimating unit 60 to perform the pulse wave estimation based on luminance information in which the measurement region is the forehead and the pulse wave estimation based on luminance information in which the measurement region is the neck independently and separately.

The pulse wave estimating unit 60 instructed to perform pulse wave estimation for each measurement region, that is, for each part of the face separates the time-series luminance signal into a pulse wave component and a noise component for each measurement region (step indicated by ST50 in FIG. 10). In the example of FIG. 9, the pulse wave estimating unit 60 separates the time-series luminance signal for the forehead into a pulse wave component and a noise component, and separates the time-series luminance signal for the neck into a pulse wave component and a noise component.

The pulse wave estimating unit 60 estimates the pulse rate for each measurement region from the time-series data of the pulse wave component extracted for each measurement region. The pulse wave estimating unit 60 according to the second embodiment calculates a most likely pulse rate from the pulse rate estimated for each measurement region. For calculation of the most likely pulse rate, for example, weighted averaging processing may be used. As the weight used in the weighted average processing, for example, reliability may be used. As the reliability, an S/N ratio of the time-series luminance signal obtained for each measurement region may be used.

As illustrated in FIG. 9, the pulse wave estimating unit 60 calculates the most likely pulse rate by using weighted average processing on the pulse rate itself, but it is not limited thereto. The pulse wave estimating unit 60 may perform weighted average processing on the luminance signal or the pulse wave component signal of the measurement region, calculate the most likely luminance signal or pulse wave component signal, and calculate the pulse rate from the plausible luminance signal or pulse wave component signal.

As described above, since the pulse wave detection device 100 according to the second embodiment has the above configuration, the influence of the phase between the plurality of time-series luminance signals of the region of interest is eliminated even when the subject wears a mask. By the operation of eliminating the influence of the phase, the pulse wave detection device 100 according to the second embodiment can detect the pulse wave signal of a subject even from an image obtained by imaging the face of the subject wearing a mask.

### Third Embodiment

In a third embodiment, the inter-region data adjusting unit 50 of the pulse wave detection device 100 according to the first embodiment is configured by two functional blocks.

In the third embodiment, the same reference numerals as those used in the above-described embodiments are used unless otherwise specified. In the third embodiment, the description overlapping with the previously described embodiment is appropriately omitted.

FIG. 11 is a block diagram illustrating a configuration of an inter-region data adjusting unit (50) of a pulse wave detection device (100) according to the third embodiment. As illustrated in FIG. 11, the inter-region data adjusting unit (50) according to the third embodiment includes a phase calculating unit 51 and a phase correcting unit 52.

The phase calculating unit 51 of the inter-region data adjusting unit 50 calculates a phase difference between a plurality of time-series luminance signals on the basis of information of a measurement region. As described above, the phase difference is a delay time appearing on the time axis.

Here, the phase difference may be obtained in such a way that the positions of peaks of two of the time-series luminance signals coincide with each other. In addition, the phase difference may be changed using the phase as a parameter, and a solution may be one in which an integral value of a square error of two of the time-series luminance signals is minimized.

The phase correcting unit 52 of the inter-region data adjusting unit 50 corrects the phase between the plurality of time-series luminance signals using the phase difference obtained by the phase calculating unit 51.

As described above, the pulse wave detection device 100 according to the third embodiment has the above configuration, and the phase between the plurality of time-series luminance signals of the region of interest is corrected even when the subject wears a mask. By the operation of correcting the phase, the pulse wave detection device 100 according to the third embodiment can detect the pulse wave signal of the subject even from an image obtained by imaging the face of the subject wearing a mask.

### INDUSTRIAL APPLICABILITY

The present disclosure technology can be applied to an on-vehicle device such as a DMS, and has industrial applicability.

### REFERENCE SIGNS LIST

10: skin region detection unit, 20: cover detection unit, 30: measurement region setting unit, 40: luminance signal extracting unit, 50: inter-region data adjusting unit, 51: phase calculating unit, 52: phase correcting unit, 60: pulse wave estimating unit, 100: pulse wave detection device, 200: camera

1. A pulse wave detector for detecting a pulse wave from an image obtained by imaging a subject, the pulse wave detector comprising:
an inter-region data adjustor including a phase calculator and a phase corrector, wherein
the phase calculator calculates a phase difference between a plurality of time-series luminance signals of a region of interest of the image, and
the phase corrector corrects a phase of the time-series luminance signal by referring to the calculated phase difference, and wherein,
the phase calculator calculates a phase by referring to an integral value of a square error of two of the time-series luminance signals.

2.-4. (canceled)
